SEQUENCE LISTING (13 sequences)

SEQ ID NO: 1 - protein, 812 aa, unidentified
  REGION 1..812: note = Streptomyces coelicolor
KIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKGENLYFQGGMASMTGGQQMGRGSGMSDITVTNWAGNITYTAKELLRPHSLDALRALVADSARVRVLGSGHSFNEIAEPGDGGVLLSLAGLPSVVDVDTAARTVRVGGGVRYAELARVVHARGLALPNMASLPHISVAGSVATGTHGSGMGNGSLASMVREVELVTADGSTVVIARGDERFGGAVTSLGALGVVTSLTLDLEPAYEMEQHVFTELPLAGLDPATFETVMAAAYSVSLFTDWRAPGFRQVWLKRRTDRPLDGFPYAAPATEKMHPVPGMPAVNCTEQFGVPGPWHERLPHFRAEFTPSSGAELQSEYLMPREHALAALHAMDAIRETLAPVLQTCEIRTVAADAQWLSPAYGRDTVAAHFTWVEDTAAVLPVVRRLEEALVPFAARPHWGKVFTVPAGELRALYPRLADFGALARALDPAGKFTNAFVRGVLAG

SEQ ID NO: 2 - protein, 560 aa, unidentified
  REGION 1..560: note = Thermosynechococcus vulcanus
MAENWRSRIITEGVQRTPNRAMLRAVGFGDEDFNKPIVGVASAHSTITPCNMGIAALASRAEAGIRAAGGMPQLFGTITVSDGISMGTEGMKYSLVSRDVIADSIETVCNAQSMDGVLAIGGCDKNMPGAMIAMARMNIPAIFVYGGTIKPGHWQGQDLTVVSAFEAVGQFSAGKMDEATLHAIEHHACPGAGSCGGMFTANTMSSAFEAMGMSLMYSSTMTAEDAEKADSTELAGKVLVEAIRKNIRPRDIITRKSIENAISVIMAVGGSTNAVLHFLAIAHSAEVPLTIDDFETIRQRVPVLCDLKPSGKYVTADLHRAGGIPQVMKMLLNAGLLHGDCLTITGETIAERLRHVPDTPDPNQDVIRPFDQPLYATGHLAILKGNLASEGAVAKISGVKNPQITGPARVFDSEEACLDAILAGKINPGDVIVIRYEGPVGGPGMREMLAPTSAIIGAGLGDSVGLITDGRFSGGTYGMVVGHVAPEAAVGGTIALVQEGDSITIDAHRRLLQLNVSEEELAARRAKWQPPAPRYTRGVLAKYAKLVSSSSLGAVTDRFV

SEQ ID NO: 3 - protein, 326 aa, unidentified
  REGION 1..326: note = Geobacillus stearothermophilus
MTTDLFTALKAKVTGTARKIVFPEGTDDRILTAASRLATEQVLQPIVLGDEQAIRVKAAALGLPLEGVEIVNPRRYGGFDELVSAFVERRKGKVTEETARELLFDENYFGTMLVYMGAADGLVSGAAHSTADTVRPALQIIKTKPGVDKTSGVFIMVRGDEKYVFADCAINIAPNSHDLAEIAVESARTAKMFGLKPRVVLLSFSTKGSASSPETEKVVEAVRLAKEMAPDLILDGEFQFDAAFVPEVAKKKAPDSVIQGDANVFIFPSLEAGNIGYKIAQRLGGFEAVGPILQGLNKPVNDLSRGCSAEDAYKLALITAAQSLGE

SEQ ID NO: 4 - protein, 393 aa, unidentified
  REGION 1..393: note = Multispecies Cupriavidus
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 5 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEGCYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTSSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 6 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFISVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 7 - protein, 315 aa, unidentified
  REGION 1..315: note = Methanosarcina mazei
MLKFSKIEKLLRNNMVSCSAPGKIYLFGEHAVVYGETAIACAVELRTRVRAELNDSITIQSQIGRTGLDFEKHPYVSAVIEKMRKSIPINGVFLTVDSDIPVGSGLGSSAAVTIASIGALNELFGFGLSLQEIAKLGHEIEIKVQGAASPTDTYVSTFGGVVTIPERRKLKTPDCGIVIGDTGVFSSTKELVANVRQLRESYPDLIEPLMTSIGKISRIGEQLVLSGDYASIGRLMNVNQGLLDALGVNILELSQLIYSARAAGAFGAKITGAGGGGCMVALTAPEKCNQVAEAIAGAGGKVTITKPTEQGLKVD

SEQ ID NO: 8 - protein, 335 aa, Streptococcus pneumoniae
MIAVKTCGKLYWAGEYAILEPGQLALIKDIPIYMRAEIAFSDSYRIYSDMFDFAVDLRPNPDYSLIQETIALMGDFLAVRGQNLRPFSLAIYGKMEREGKKFGLGSSGSVVVLVVKALLALYNLSVDQNLLFKLTSAVLLKRGDNGSMGDLACIAAEDLVLYQSFDRQKVAAWLEEENLATVLERDWGFSISQVKPTLECDFLVGWTKEVAVSSHMVQQIKQNINQNFLTSSKETVVSLVEALEQGKSEKIIEQVEVASKLLEGLSTDIYTPLLRQLKEASQDLQAVAKSSGAGGGDCGIALSFDAQSTKTLKNRWADLGIELLYQERIGHDDKS

SEQ ID NO: 9 - protein, 344 aa, Streptococcus pneumoniae
MYHSLGNQFDTRTRTSRKIRRERSCSDMDREPVTVRSYANIAIIKYWGKKKEKEMVPATSSISLTLENMYTETTLSPLPANVTADEFYINGQLQNEVEHAKMSKIIDRYRPAGEGFVRIDTQNNMPTAAGLSSSSSGLSALVKACNAYFKLGLDRSQLAQEAKFASGSSSRSFYGPLGAWDKDSGEIYPVETDLKLAMIMLVLEDKKKPISSRDGMKLCVETSTTFDDWVRQSEKDYQDMLIYLKENDFAKIGELTEKNALAMHATTKTASPAFSYLTDASYEAMDFVRQLREKGEACYFTMDAGPNVKVFCQEKDLEHLSEIFGQRYRLIVSKTKDLSQDDCC

SEQ ID NO: 10 - protein, 182 aa, unidentified
  REGION 1..182: note = Multispecies Bacteria
MQTEHVILLNAQGVPTGTLEKYAAHTADTRLHLAFSSWLFNAKGQLLVTRRALSKKAWPGVWTNSVCGHPQLGESNEDAVIRRCRYELGVEITPPESIYPDFRYRATDPSGIVENEVCPVFAARTTSALQINDDEVMDYQWCDLADVLHGIDATPWAFSPWMVMQATNREARKRLSAFTQLK

SEQ ID NO: 11 - protein, 297 aa, unidentified
  REGION 1..297: note = Geobacillus stearothermophilus
MAQLSVEQFLNEQKQAVETALSRYIERLEGPAKLKKAMAYSLEAGGKRIRPLLLLSTVRALGKDPAVGLPVACAIEMIHTYFLIHDDLPSMDNDDLRRGKPTNHKVFGEAMAILAGDGLLTYAFQLITEIDDERIPPSVRLRLIERLAKAAGPEGMVAGQAADMEGEGKTLTLSELEYIHRHKTGKMLQYSVHAGALIGGADARQTRELDEFAAHLGLAFQIRDDILDIEGAEEKIGKPVGSDQSNNKATYPALLSLAGAKEKLAFHIEAAQRHLRNADVDGAALAYICELVAARDH

SEQ ID NO: 12 - protein, 300 aa, unidentified
  REGION 1..300: note = Rhizobacteria
MALDEAPAEARPGSRAVELEIDGRSRIFDIDDPDLPKWIDEEAFRSDDYPYKKKLDREEYEETLTKLQIELVKVQFWMQATGKRVMAVFEGRDAAGKGGAIHATTANMNPRSARVVALTKPTETERGQWYFQRYVATFPTAGEFVLFDRSWYNRAGVEPVMGFCTPDQYEQFLKEAPRFEEMIANEGIHLFKFWINIGREMQLKRFHDRRHDPLKIWKLSPMDIAALSKWDDYTGKRDRMLKETHTEHGPWAVIRGNDKRRSRINVIRHMLTKLDYDGKDEAAIGEVDEKILGSGPGFLR

SEQ ID NO: 13 - protein, 261 aa, unidentified
  REGION 1..261: note = Bacillus sp. G3
MYSDLEGKVVVITGSASGLGRAMGVRFAREKAKVVINYRSRESEANDVLEEIKKVGGEAIAVKGDVTVESDVVNLIQSAVKEFGTLDVMINNAGIENAVPSHEMPLEDWNRVINTNLTGAFLGSREAIKYFVEHDIKGSVINMSSVHEKIPWPLFVHYAASKGGMKLMTETLAMEYAPKGIRVNNIGPGAINTPINAEKFADPKKRADVESMIPMGYIGKPEEIAAVATWLASSEASYVTGITLFADGGMTLYPSFQAGRG

(19) United States
(12) Patent Application Publication
(10) Pub. No.: US 2025/0207160 A1
(43) Pub. Date: Jun. 26, 2025

(54) CELL-FREE PRODUCTION OF GERANYL PYROPHOSPHATE FROM GLYCEROL IN A CELL-FREE MANUFACTURING SYSTEM

(71) Applicant: DEBUT BIOTECHNOLOGY, INC., San Diego, CA (US)

(72) Inventors: Joshua Britton, San Diego, CA (US); Nicholas Brideau, San Diego, CA (US)

(21) Appl. No.: 18/677,251

(22) Filed: May 29, 2024

Related U.S. Application Data
(63) Continuation of application No. 17/554,537, filed on Dec. 17, 2021, now Pat. No. 12,006,528.
(60) Provisional application No. 63/127,836, filed on Dec. 18, 2020.
(60) Provisional application No. 63/127,758, filed on Dec. 18, 2020.

(51) Int. Cl. C12P 9/00; B01L 3/00; C12P 7/42

(52) U.S. Cl. CPC C12P 9/00; B01L 3/00; C12P 7/42; B01L 2200/028; B01L 2200/0631; B01L 2200/0689; B01L 2200/146; B01L 2200/147; B01L 2300/048; B01L 2300/0663; B01L 2300/0832; B01L 2300/163; B01L 2300/1894

(57) ABSTRACT

Geranyl pyrophosphate (GPP) is a key intermediate molecule in the bioproduction of thousands of natural products. Currently, natural products are either cultivated from plants, synthesized via complex chemical synthesis strategies, or through cell-based factories also known as biofoundries. However, in order to replicate the process in a cell free environment, numerous enzymes and cofactors must be utilized making this approach costly and unviable. In order to make this process viable, a new approach was needed that uses fewer enzymes and cofactors. As described herein, the present invention demonstrates that it is possible to create GPP from glycerol through a short and concise biosynthetic pathway outside of the cell.

### CROSS-REFERENCES TO RELATED APPLICATIONS

This application is a non-provisional and claims benefit of U.S. Provisional Application No. 63/127,836 filed Dec. 18, 2020 and of U.S. Provisional Application No. 63/127,758 filed Dec. 18, 2020, the specification of which is incorporated herein in their entirety by reference.

### REFERENCE TO A SEQUENCE LISTING

Applicant asserts that the paper copy of the Sequence Listing is identical to the Sequence Listing in computer readable form found on the accompanying computer file, entitled DEBUT_20_03_NP_Sequence_Listing_ST25. The content of the sequence listing is incorporated herein by reference in its entirety.

### FIELD OF THE INVENTION

The present invention features a method of producing geranyl pyrophosphate (GPP) from glycerol, in particular, the present invention features a cell-free production method.

### BACKGROUND OF THE INVENTION

Geranyl pyrophosphate (GPP) is a key intermediate molecule in the bioproduction of thousands of natural products. Here, a natural product is a molecule formed through multi-step enzyme-pathways in organisms such as plants, animals, and bacteria. Currently, natural products are either cultivated from plants, synthesized via complex chemical synthesis strategies, or through cell-based factories also known as biofoundries.

Previously, Valliere et. al. 2019 manufactured GPP in a cell free environment using glucose as a starting material. This complete replica of a natural enzyme system uses 22 enzymes and requires 40 cofactor equivalents including 12 adenosine triphosphate (ATP), 12 adenosine diphosphate (ADP), four nicotinamide adenine dinucleotide phosphate (NADP⁺), two nicotinamide adenine dinucleotides (NAD⁺), six acetyl-coenzyme A (CoA), and four nicotinamide adenine dinucleotide phosphate (NADPH). The number of cofactors required by the process described in the current literature means that this approach will always be costly and unviable (FIG. 1B). In order to make this process viable, a new approach is needed that uses fewer enzymes and cofactors and improves reliability. As described herein, the present invention demonstrates that it is possible to create GPP through a short and concise biosynthetic pathway outside of the cell; known as cell-free biosynthesis.

### BRIEF SUMMARY OF THE INVENTION

It is an objective of the present invention to provide a cell-free method that allows for the production of producing geranyl pyrophosphate (GPP) and additional secondary metabolites starting from glycerol via a cell-free biosynthesis platform, as specified in the independent claims. Embodiments of the invention are given in the dependent claims. Embodiments of the present invention can be freely combined with each other if they are not mutually exclusive.

Manufacturing natural products via cultivation, chemical synthesis, or in the cell suffers from many problems that limit the commercial viability of high-value chemical production. First, cultivation is often economically unfeasible, requires a vast amount of land/energy/water and the plant is only capable of producing the high-value material in low amounts. Next, chemical synthesis requires extensive, elaborate, expensive, toxic, and inefficient multi-step chemical reactions to produce natural products that often are too complex to make in the laboratory. Finally, bio-foundries (use of the whole cell) suffer from product toxicity, carbon flux redirection, diffusion problems through cell walls, and toxic byproduct generation. To avoid these above problems, cell-free manufacturing presents as a viable alternative.

In cell-free systems, the key components of the cell namely cofactors and enzymes are used in a chemical reaction without the cell. The same enzymes that are found in plants, animals, and bacteria are created in vivo (typically through protein overexpression in hosts such as bacteria), isolated via chromatography, and then added into a bioreactor with a substrate (starting material). The enzymes transform the substrate in the same way that occurs in plants, animals, and bacteria, but without the complexity of the organism. In this way, natural products can begin to be created without the plant, cell, or chemical synthesis.

The present invention creates an enzyme pathway that removes the glycolysis pathway (glucose to pyruvic acid) to allow a shorter, affordable, and simpler process to convert glycerol into GPP (FIG. 1A). The presently claimed process uses 12 enzymatic steps and eliminates 24 cofactor equivalents and ten enzymes (FIG. 1B). Additionally, enzyme immobilization was used to improve conversion to GPP by avoiding enzyme precipitation, inactivation, and the unreliability of the non-immobilized system. The present invention also provides evidence that demonstrates that without enzyme immobilization, enzyme precipitation and destruction of the enzyme pathway is observed. Finally, this process starts with glycerol and not glucose. Glycerol is an abundant natural material that is not used in the production of GPP; this invention allows this natural waste product to be utilized as a viable alternative to glucose.

In some embodiments, the present features a method of converting glycerol to geranyl pyrophosphate (GPP) and additional secondary metabolites. In some embodiments, the method comprises adding glycerol to a reaction mixture. In some embodiments, the method comprises adding a plurality of enzymes to the aforementioned reaction mixture. In some embodiments, the enzymes are selected from a group consisting of alditol oxidase (Aldo), dihydroxy-acid dehydratase (DHAD), pyruvate oxidase (PyOx), acetyl-phosphate transferase (PTA), acetyl-CoA acetyltransferase (PhaA), HMG-COA Synthase A110G (HMGS), HMG-COA Reductase (HMGR), mevalonate kinase (MVK), phosphomevalonate kinase (PMVK), diphosphomevalonate kinase (MDC), isopentyl-PP Isomerase (IDI), and farnesyl-PP synthase S82F (FPPS). In some embodiments, the method comprises removing a supernatant from the aforementioned reaction mixture. In some embodiments, the method comprises isolating or producing GPP. In some embodiments, the enzymes may be added to the reaction mixture asynchronously. In other embodiments, the enzymes may be added to the reaction mixture simultaneously.

In other embodiments, the present invention features a method of converting glycerol to geranyl phosphate (GPP) and additional secondary metabolites. In some embodiments, the method comprises adding glycerol and alditol oxidase (Aldo) to a reaction mixture. In some embodiments, the method further comprises adding dihydroxy-acid dehydratase (DHAD) to the reaction mixture. In some embodiments, the method comprises removing a supernatant of the aforementioned reaction mixture and adding pyruvate oxidase (PyOx) to the supernatant of the reaction mixture. In some embodiments, the method comprises removing a supernatant of the aforementioned reaction mixture and at least two enzymes selected from a group consisting of acetyl-phosphate transferase (PTA), acetyl-CoA acetyltransferase (PhaA), HMG-COA Synthase A110G (HMGS), HMG-CoA Reductase (HMGR), mevalonate kinase (MVK), phosphomevalonate kinase (PMVK), diphosphomevalonate kinase (MDC), isopentyl-PP Isomerase (IDI), and farnesyl-PP synthase S82F (FPPS) to the supernatant of the reaction mixture. In some embodiments, the method comprises removing a supernatant from the aforementioned reaction mixture and producing GPP. In some embodiments, the enzymes may be added to the reaction mixture asynchronously. In other embodiments, the enzymes may be added to the reaction mixture simultaneously.

One of the unique and inventive technical features of the present invention is the use of a cell-free system for the production of geranyl pyrophosphate (GPP) and additional secondary metabolites starting from glycerol. Without wishing to limit the invention to any theory or mechanism, it is believed that the technical feature of the present invention advantageously provides for higher reaction concentrations, no cell-wall to battle for product and substrate diffusion, no competition in the cell for carbon flux and byproduct formation, no cell death due to the formation of toxic compounds (there is no cell), and increased flexibility as a platform solution for creating a large number of compounds compared to cells having to be re-programmed every time. In this approach, one may simply change an enzyme in the pathway to create a new chemical entity.

Furthermore, the prior references teach away from the present invention. For example, Valliere et. al. 2019 used a more complex cell-free system to generate GPP as an intermediate in the production of cannabidiol (CBD) from glucose. The pathway of the present invention eliminates 60% of the expensive cofactors, ten enzymes, and an elaborate purge valve and uses an alternative starting material. While the present invention shares 10 enzymes with the Valliere et. al. approach, the present invention has significantly improved the enzyme activity, longevity and reliability by immobilizing and optimizing all 10 enzymes. Of note, Valliere et. al. contains data demonstrating enzyme precipitation following incubation of all enzymes in a reaction mixture. Precipitation of these enzymes was also seen when repeating the work in as little as 16 hours after being combined in a reaction mixture, severely limiting the previous work. The key success was the immobilization of each of the 12 enzymes to allow the enzymes to remain active in reaction mixtures for extended periods of time via eliminating precipitation.

Furthermore, the inventive technical features of the present invention contributed to a surprising result. The first module in the pathway of the present invention converts glycerol to pyruvic acid by combining the activity of alditol oxidase (ALDO) and dihydroxy acid dehydratase (DHAD) as reported in Gao et. al. 2015. Surprisingly, the present invention had to make several modifications to this published work in order to successfully generate pyruvic acid from glycerol. First, a maltose binding protein was added to the N-terminus of Streptomyces ALDO to aid in solubility and stability while maintaining activity of the enzyme (SEQ ID NO: 1). Second, it was found that MBP-ALDO required oxygenation for optimal activity. Third, Sulfolobus solfataricus DHAD as reported in Gao et. al. 2015 was not functional. Many DHAD orthologs were screened and Thermosynechococcus vulcanus DHAD (SEQ ID NO: 2) was found to be the most active. Fourth, it was found that DHAD required deoxygenation and a very specific pH range for optimal activity.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B show the pathway used for the production of geranyl pyrophosphate (GPP), (left, FIG. 1A) vs. previous attempts to GPP from glucose (right, FIG. 1B). As shown the pathway on the left (FIG. 1A) has 70% fewer co-factors and 10 fewer enzymes.

FIGS. 2A and 2B show HPLC traces for cannabigerolic acid (CBGA) for both CBGA standards (top, FIG. 2A) and immobilized enzyme batch reactions (bottom, FIG. 2B). The retention time of 3.68 minutes at 228 nm is noted for both reactions.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.

As used herein, “reaction solution” may refer to all components necessary for enzyme-based chemical transformation. This is typically, but not limited to, buffering agent, salts, cofactor, and substrate (i.e., starting material).

As used herein, “reaction mixture” may refer to all components from the “reaction solution” plus the enzyme(s) and/or products from the reaction. In some embodiments, the “reaction mixture” may refer to just the reaction solution without any enzymes or reaction products.

In some embodiments, “reaction solution” and “reaction mixture” may be used interchangeably.

As used herein, “buffering agents” may refer to chemicals added to water-based solutions that resist changes in pH by the action of acid-base conjugate components.

As used herein, “supernatant” may refer to the soluble liquid fraction of a sample.

As used herein, “batch reactions” may refer to a chemical or biochemical reaction performed in a closed system such as a fermenter or typical reaction flask.

As used herein, “cofactors” may refer to a non-protein chemical compound that may bind to a protein and assist with a biological chemical reaction. Co-factors may be metal ions, organic compounds, or other chemicals. Non-limiting examples of cofactors may include but are not limited to ATP and NADPH.

As used herein, “cofactor recycling” may refer to regeneration of functional cofactor capable of participating in enzyme-catalyzed reactions. A non-limiting example of this regeneration is a separate reaction acting on the altered cofactor produced by a primary enzymatic reaction, such as the enzymatic conversion of ADP back to ATP.

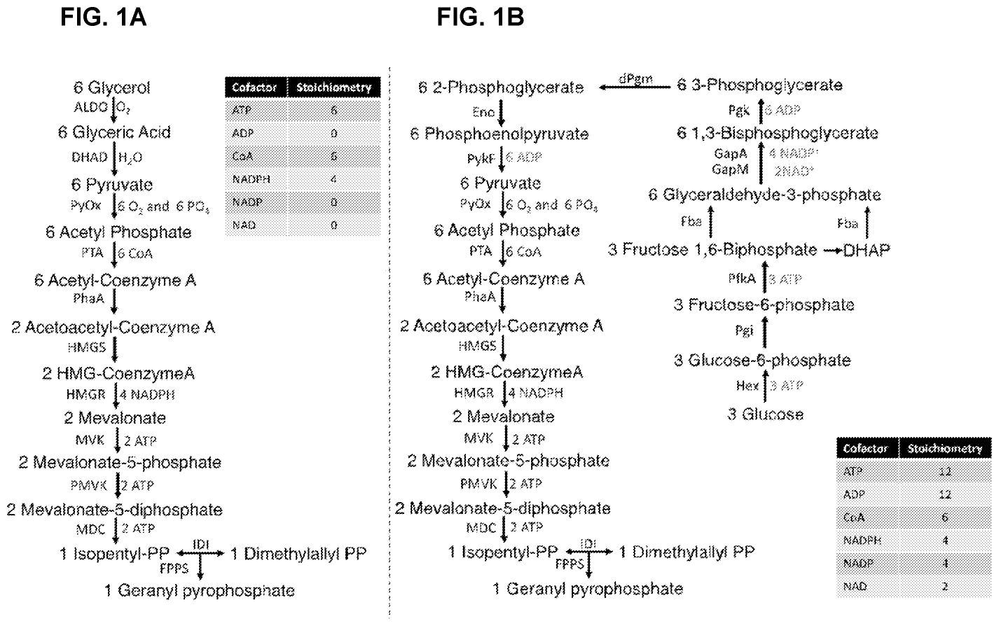

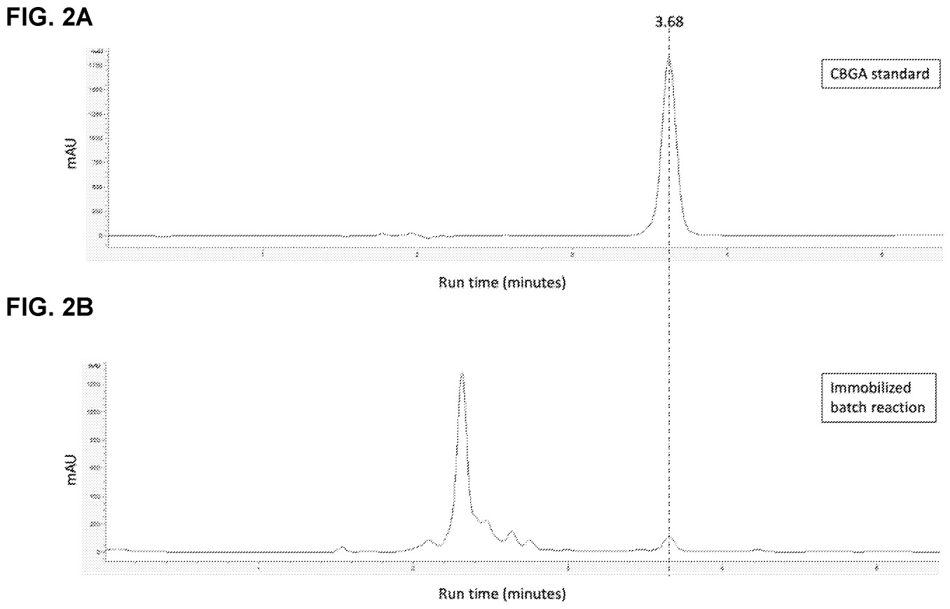

Referring now to FIGS. 1A, 1B, 2A, and 2B, the present invention features a method of producing geranyl pyrophosphate (GPP) and additionally secondary metabolites from glycerol.

The present features a method of converting glycerol to geranyl pyrophosphate (GPP) and additional secondary metabolites. In some embodiments, the method comprises adding glycerol to a reaction mixture. In some embodiments, the method comprises adding a plurality of enzymes to the aforementioned reaction mixture. In some embodiments, the enzymes are selected from a group consisting of alditol oxidase (Aldo), dihydroxy-acid dehydratase (DHAD), pyruvate oxidase (PyOx), acetyl-phosphate transferase (PTA), acetyl-CoA acetyltransferase (PhaA), HMG-COA Synthase A110G (HMGS), HMG-COA Reductase (HMGR), mevalonate kinase (MVK), phosphomevalonate kinase (PMVK), diphosphomevalonate kinase (MDC), isopentyl-PP Isomerase (IDI), and farnesyl-PP synthase S82F (FPPS). In some embodiments, the method comprises removing a supernatant from the aforementioned reaction mixture. In some embodiments, the method comprises isolating or producing GPP.

The present invention may also feature a method of converting glycerol to geranyl phosphate (GPP) and additional secondary metabolites. In some embodiments, the method comprises adding glycerol and alditol oxidase (Aldo) to a reaction mixture. In some embodiments, the method further comprises adding dihydroxy-acid dehydratase (DHAD) to the reaction mixture. In some embodiments, the method comprises removing a supernatant of the aforementioned reaction mixture and adding pyruvate oxidase (PyOx) to the supernatant of the reaction mixture. In some embodiments, the method comprises removing a supernatant of the aforementioned reaction mixture and at least two enzymes selected from a group consisting of acetyl-phosphate transferase (PTA), acetyl-CoA acetyltransferase (PhaA), HMG-COA Synthase A110G (HMGS), HMG-COA Reductase (HMGR), mevalonate kinase (MVK), phosphomevalonate kinase (PMVK), diphosphomevalonate kinase (MDC), isopentyl-PP Isomerase (IDI), and farnesyl-PP synthase S82F (FPPS) to the supernatant of the reaction mixture. In some embodiments, the method comprises removing the supernatant from the aforementioned reaction mixture and producing or isolating GPP.

In some embodiments, at least one enzyme is added to the reaction mixture. In some embodiments, at least two enzymes are added to the reaction mixture. In some embodiments, at least three enzymes are added to the reaction mixture. In some embodiments, at least four enzymes are added to the reaction mixture. In some embodiments, at least five enzymes are added to the reaction mixture. In some embodiments, at least six enzymes are added to the reaction mixture. In some embodiments, at least seven enzymes are added to the reaction mixture. In some embodiments, at least eight enzymes are added to the reaction mixture. In some embodiments, at least nine enzymes are added to the reaction mixture. In some embodiments, at least ten enzymes are added to the reaction mixture. In some embodiments, at least eleven enzymes are added to the reaction mixture. In some embodiments, at least twelve enzymes are added to the reaction mixture.

In some embodiments, the enzymes may be added to the reaction mixture asynchronously. In other embodiments, the enzymes may be added to the reaction mixture simultaneously.

In some embodiments, the method further comprises adding a NphB enzyme before the final removal of the supernatant from the reaction mixture to convert GPP to cannabigerolic acid (CBGA). In some embodiments, CBGA is used to determine the amount of GPP produced in the above-mentioned method. In some embodiments, the production of CBGA is used as an analytical tool. In some embodiments, the production of CBGA from GPP by NphB is used as a detection method. In some embodiments, the production of CBGA by NphB is used to detect the amount of GPP. In some embodiments, the amount of CBGA produced from the conversion of GPP by the NphB enzyme is 1:1.

In some embodiments, the reaction mixtures described herein comprise cofactors. In some embodiments, the cofactors comprise adenosine triphosphate (ATP), nicotinamide adenine dinucleotide (NAD⁺), nicotinamide adenine dinucleotide phosphate (NADP⁺), or a combination thereof. In some embodiments, the methods described herein utilized cofactor recycling. In some embodiments, the cofactors are recycled. In some embodiments, the method further comprises adding glucose dehydrogenase (GDH), and polyphosphate kinase 2 (PPK2) to the reaction mixture. In some embodiments, glucose dehydrogenase (GDH), and polyphosphate kinase 2 (PPK2) are added to the reaction mixture to recycle the cofactors.

In some embodiments, the temperature of the reaction may range from about 22° C. to about 50° C. In some embodiments, the temperature of the reaction is about 20° C. In some embodiments, the temperature of the reaction is about 25° C. In some embodiments, the temperature of the reaction is about 30° C. In some embodiments, the temperature of the reaction is about 35° C. In some embodiments, the temperature of the reaction is about 40° C. In some embodiments, the temperature of the reaction is about 45° C. In some embodiments, the temperature of the reaction is about 50° C. In some embodiments, the temperature of the reaction is about 55° C.

In some embodiments, the pH of the reaction may range from about 6.5 to about 9.0. In some embodiments, the pH of the reaction is about 5.0. In some embodiments, the pH of the reaction is about 5.5. In some embodiments, the pH of the reaction is about 6.0. In some embodiments, the pH of the reaction is about 6.5. In some embodiments, the pH of the reaction is about 7.0. In some embodiments, the pH of the reaction is about 8.5. In some embodiments, the pH of the reaction is about 9.0. In some embodiments, the pH of the reaction is about 9.5. In some embodiments, the pH of the reaction is about 10.0.

In some embodiments, the time to run the reaction may range from about 2 hours to about 32 hours. In some embodiments, the time to run the reaction is about 0.5 hour. In some embodiments, the time to run the reaction is about 1 hour. In some embodiments, the time to run the reaction is about 2 hours. In some embodiments, the time to run the reaction is about 5 hours. In some embodiments, the time to run the reaction is about 10 hours. In some embodiments, the time to run the reaction is about 15 hours. In some embodiments, the time to run the reaction is about 20 hours. In some embodiments, the time to run the reaction is about 25 hours. In some embodiments, the time to run the reaction is about 30 hours. In some embodiments, the time to run the reaction is about 35 hours. In some embodiments, the time to run the reaction is about 40 hours. In some embodiments, the time to run the reaction is about 45 hours. In some embodiments, the time to run the reaction is greater than 45 hours.

In some embodiments, the enzymes are immobilized. In some embodiments, immobilized enzymes are immobilized onto solid supports. Non-limiting examples of solid supports may include but are not limited to epoxy methacrylate, amino C₆ methacrylate, or microporous polymethacrylate. In further embodiments, various surface chemistries may be used for linking the immobilized enzyme to a solid surface, including but not limited to covalent, adsorption, ionic, affinity, encapsulation, or entrapment. In other embodiments, the enzymes are non-immobilized.

In some embodiments, one or more of the enzymes are immobilized. In other embodiments, all the enzymes are immobilized. In some embodiments, one or more of the enzymes are non-immobilized. In other embodiments, all the enzymes are non-immobilized. In some embodiments, the plurality of enzymes are immobilized. In other embodiments, the plurality of enzymes are non-immobilized.

In some embodiments, various reaction conditions may be altered to ensure functional enzymes, including but not limited to reaction time, oxygenation/deoxygenation, pH, buffering agents, and reaction temperature.

In some embodiments, the methods described herein teaches away from previously described methods because the presently claimed methods utilized less enzymes and cofactors to produce GPP. In certain embodiments, methods described herein do not use 24 cofactor equivalents used by Valliere et al., (or 60% of cofactors). In certain embodiments, only ten of the enzymes taught by Valliere et al. are used in the presently claimed method.

### Example

The following is a non-limiting example of the present invention. It is to be understood that said example is not intended to limit the present invention in any way. Equivalents or substitutes are within the scope of the present invention.

Enzyme Expression and Purification: All genes were synthesized and cloned into expression plasmids and then transformed into E. coli cells for expression. Cells were grown in TB media supplemented with 50 μg/mL kanamycin sulfate at 37° C. and 200 rpm until A₆₀₀=0.6. Cells were cooled to 18° C., expression was induced and grown for an additional 18 h. Cell pellets were collected by centrifugation, frozen, and then resuspended in a 5 mL lysis buffer (50 mM Tris pH 8.0, 300 mM NaCl, 5% glycerol, 1 mM PMSF) per gram of cell paste. Cell lysates were prepared by sonication and cellular debris was removed by centrifugation. Clarified lysate was loaded onto GE XK series columns containing IMAC-Nickel resin. Proteins were eluted using a 15CV gradient from buffer A (50 mM Tris pH 8.0, 300 mM NaCl, 10% glycerol) into 70% buffer B (2 M imidazole, 50 mM Tris pH 8.0, 300 mM NaCl, 10% glycerol). Fractions containing proteins of interest were pooled and transitioned into buffer A (above) with a GE HiPrep 26/10 desalting column, with the exception of MBP-Aldo, which was stored in 50 mM Tris pH 8.0, 500 mM NaCl, 0.1% Triton X-100.

1.0 Production of GPP with the non-immobilized cell-free pathway: As described herein, the pathway first converts glycerol to glyceric acid using alditol oxidase (Aldo, EC 1.1.3.41) before subsequent conversion into pyruvic acid using dihydroxy-acid dehydratase (DHAD, EC 4.2.1.9, FIG. 1A). While the first two enzymes have been documented, several significant advancements were required to make this pathway functional, as the previously reported work could not be replicated.

To ensure functional enzymes, specific reaction conditions including timed reaction oxygenation, de-oxygenation, pH changes, specific buffers, and reaction temperature had to be found. First, the reported Aldo is unstable and inactive. To overcome this, a modified Aldo was created housing a fusion maltose-binding protein (MBP) tag to improve solubility and stability in solution (SEQ ID 1). The MBP-fused Aldo converted glycerol into glyceric acid (100%, 1.75 g/L). Second, the published reaction with Sulfolobus solfataricus DHAD was also not reproducible. To overcome this, many DHAD enzyme orthologs were screened; and it was found that DHAD from Thermosynechococcus vulcanus converted 100% of glyceric acid to pyruvic acid (1.32 g/L).

With these new enzymes, a one-pot reaction containing both Aldo (11 μM) and DHAD (16 μM) converted glycerol into pyruvic acid (100% conversion, yield 1.23 g/L). Compared to previous work starting from glucose, the present improved system afforded 14 mM of pyruvic acid in 24 hours, without the use of cofactors and nine fewer enzymes providing a significant improvement. It should also be noted that previous attempts from glucose require many additional enzymes, cofactors, and reaction manufacturing complications such as protein precipitation. Removing these constraints allows a commercially viable approach to GPP from an inexpensive carbon source.

After optimization of Aldo and DHAD, the remaining enzymes in the pathway that convert pyruvic acid into geranyl pyrophosphate had to be created and optimized (GPP, FIGS. 1A and 1B). After optimization of each individual enzyme, GPP was afforded (43 mg/L in 120 hours). This result validated the shorter improved pathway with far fewer cofactors for GPP manufacturing, however, protein precipitation was observed after several days meaning that this approach would not be suitable for commercial production. To overcome this limitation, each protein in the pathway was immobilized onto solid supports to ensure the protein remained active and operational; this required extensive optimization and understanding to create each individual enzyme-solid support complex.

2.0 Protein Immobilization and Optimization: To increase stability, longevity, and catalysis, each purified enzyme in the pathway was immobilized onto solid supports. Different commercial support materials were routinely screened for product and substrate retention, enzyme retention, and activity of the immobilized enzyme. The support collection comprised of various surface chemistries for the following types of linkage: covalent, adsorption, ionic, affinity, encapsulation, and entrapment. Typically, 50 mg of resin was mixed with 4.0 mg of an enzyme in a desalting buffer 16-24 h at room temperature. The amount of immobilized enzyme was quantified by measuring protein concentration in solution before and after immobilization by either BCA or Bradford assay. All immobilized enzymes were screened for optimal values for resin type, substrate concentration (5 mM-250 mM), pH (5.0-9.0), temperature (20-50° C.), buffering agent (Tris, HEPES, PO₄), and time (1 h-36 h). Optimal reaction conditions and results for each enzyme are as follows (Table 1A).

TABLE 1A
Conditions found for the immobilized enzymes used in the GPP
biomanufacturing route (FIG. 1A).

| | Reaction Conditions | | | |
|---|---|---|---|---|
| | Screened | | | |
| | Temp | | Time | Yield |
| Enzyme Name | (° C.) | pH | (h) | (%) |
| MBP-Aldo (Aldo) | 37 | 9.0 | 21 | 98 |
| Dihydroxy Acid Dehydratase (DHAD) | 45 | 8.0 | 16 | 32 |
| Pyruvate Oxidase (PyOx) | 37 | 6.5 | 16 | 91 |
| Acetyl-phosphate transferase (PTA) | 32 | 8.0 | 8 | 60 |
| Acetyl-CoA acetyltransferase (PhaA) | 32 | 8.0 | 8 | 44 |
| HMG-CoA Synthase A110G (HMGS) | 32 | 7.5 | 2 | 54 |
| HMG-CoA Reductase (HMGR) | 37 | 7.0 | 2 | 98 |
| Mevalonate Kinase (MVK) | 37 | 8.0 | — | 87 |
| Phosphomevalonate Kinase (PMVK) | 37 | 8.0 | 32 | 96 |
| Diphosphomevalonate Kinase (MDC) | 37 | 8.0 | 16 | 94 |
| Isopentyl-PP Isomerase (IDI) | 22 | 8.0 | 2 | 28 |
| Farnesyl-PP synthase S82F (FPPS) | 25 | 8.3 | 4 | 81 |
| Prenyl transferase (NphB) | 50 | 8.0 | 6 | 16 |

The percent yields in the foregoing table are presented for illustrative purposes. In some embodiments, each step of the GPP biomanufacturing process may have a percent yield of up to 99%, up to 99.5%, up to 99.9% or up to 100%. In some embodiments, the percent yields in each step of the GPP biomanufacturing process may have values within the ranges in the following table (Table 1B).

TABLE 1B
Exemplary ranges for the immobilized enzymes that may be used
in the GPP biomanufacturing route (FIG. 1A).

| | Exemplary Reaction | | | |
|---|---|---|---|---|
| | Conditions | | | |
| | Approxi- | Approxi- | Approxi- | |
| | mate | mate | mate | Yield |
| Enzyme Name | Temp (° C.) | pH | Time (h) | (%) |
| MBP-Aldo (Aldo) | 37 | 9 | 21 | 98-100% |
| Dihydroxy Acid | 45 | 8.0 | 16 | 32-100% |
| Dehydratase (DHAD) | | | | |
| Pyruvate Oxidase (PyOx) | 37 | 6.5 | 16 | 91-100% |
| Acetyl-phosphate | 32 | 8.0 | 8 | 60-100% |
| transferase (PTA) | | | | |
| Acetyl-CoA | 32 | 8.0 | 8 | 44-100% |
| acetyltransferase (PhaA) | | | | |
| HMG-CoA Synthase | 32 | 7.5 | 2 | 54-100% |
| A110G (HMGS) | | | | |
| HMG-CoA Reductase | 37 | 7.0 | 2 | 98-100% |
| (HMGR) | | | | |
| Mevalonate Kinase | 37 | 8.0 | — | 87-100% |
| (MVK) | | | | |
| Phosphomevalonate | 37 | 8.0 | 32 | 96-100% |
| Kinase (PMVK) | | | | |
| Diphosphomevalonate | 37 | 8.0 | 16 | 94-100% |
| Kinase (MDC) | | | | |
| Isopentyl-PP Isomerase | 22 | 8.0 | 2 | 28-100% |
| (IDI) | | | | |
| Farnesyl-PP synthase | 25 | 8.3 | 4 | 81-100% |
| S82F (FPPS) | | | | |
| Prenyl transferase (NphB) | 50 | 8.0 | 6 | 16-100% |

2.1 Optimization of Alditol Oxidase (Aldo): MBP-Aldo was immobilized onto activated amino C₆ methacrylate resin. The immobilized enzyme was used to convert glycerol into glyceric acid. The reaction solution (50 mM Tris pH 9, 2.5 mM MgCl₂, 20 mM glycerol) was mixed with 50 UM immobilized enzyme at 37° C. for 21 hours. Immobilized MBP-Aldo converted 100% of 20 mM glycerol to yield 20 mM (1.75 g/L) glyceric acid. For sampling, the reaction mixture was analyzed through high-performance liquid chromatography (HPLC). The HPLC method was as follows: An Agilent 1200 HPLC was fitted with a 30 cm Aminex HPX-87H column equipped with a micro-guard cation H refill cartridge. The column was heated to 55° C. with the sample block being maintained at 25° C. For each sample, 1 L was injected, and using a mobile phase comprised of 100% sulfuric acid (10 mM). The sample time was a total of 45 minutes with glyceric acid eluting at 17.2 mins and glycerol eluting at 21.0 minutes. A Refractive Index Detector (RID, Agilent) was used after a 2 h equilibration period produced a stable baseline.

2.2 Optimization of Dihydroxy Dehydratase (DHAD): DHAD was mixed with activated amino C₆ methacrylate resin and the immobilized enzyme was used to convert glyceric acid into pyruvic acid. The reaction solution (50 mM Tris pH 8.5, 2.5 mM MgCl₂, 20 mM glyceric acid) was mixed with 50 μM immobilized enzyme at 45° C. for 16 hours. Immobilized DHAD was able to convert 99% of 20 mM glyceric acid for a yield of 15 mM (1.32 g/L, 75%) pyruvic acid. For sampling, the reaction mixture was examined on an HPLC system to examine the amount of glycerol and glyceric acid. The HPLC method was as follows: An Agilent 1200 HPLC was fitted with a 30 cm Aminex HPX-87H column equipped with a micro-guard cation H refill cartridge. The column was heated to 55° C. with the sample block being maintained at 25° C. For each sample, 1 μL was injected and an isocratic gradient comprised of 100% sulfuric acid (10 mM) was used as the mobile phase. The sample time was a total of 45 minutes with pyruvic acid eluting at 16.0 minutes and glyceric acid eluting at 17.2 mins. A Refractive Index Detector (Agilent) was used after a 2 h equilibration period produced a stable baseline.

2.3 Optimization of Pyruvate Oxidase (PyOx): PyOx was mixed with activated amino C₆ methacrylate resin and the immobilized enzyme was used to convert pyruvic acid into acetyl phosphate. The reaction solution (10 mM Tris, 50 mM KH₂PO₄, 50 mM K₂HPO₄, PH 6.5, 5.0 mM MgCl₂, 100 mM NaCl, 20 mM pyruvic acid, 20 mM thiamine pyrophosphate) was mixed with 3.85 μM immobilized enzyme at 37° C. for 16 hours. Immobilized PyOx was able to convert 91% of 5 mM pyruvate for a yield of 4.55 mM (837 mg/L) acetyl phosphate. For sampling, the reaction fluid was examined on an HPLC system to examine the amount of pyruvate and acetyl phosphate. The HPLC method was as follows: An Agilent 1200 HPLC was fitted with a 30 cm Aminex HPX-87H column equipped with a micro-guard cation H refill cartridge. The column was heated to 55° C. with the sample block being maintained at 25° C. The HPLC method comprised of 5 μl sample injection volume and an isocratic gradient comprised of 100% sulfuric acid (10 mM) was used as the mobile phase. The run time was a total of 25 minutes with acetyl phosphate eluting at 23.6 mins and pyruvate eluting at 16.0 minutes. A Refractive Index Detector (Agilent) was used after a 2 h equilibration period produced a stable baseline.

2.4 Optimization of Phosphate acetyltransferase (PTA): PTA was mixed with epoxy methacrylate resin and the immobilized enzyme was used to convert acetyl phosphate into acetyl-coenzyme A (acetyl-CoA). The reaction solution (10 mM Tris, 50 mM KH₂PO₄, 50 mM K₂HPO₄, pH 8.0, 5.0 mM MgCl₂, 100 mM NaCl, 3.2 mM acetyl phosphate, 3.2 mM CoA) was mixed with immobilized enzyme at 32° C. for 8 hours. Immobilized PTA (38.4 μM) was able to convert 60% of 3.2 mM acetyl phosphate for a yield of 1.92 mM (1.7 g/L) acetyl-CoA. For sampling, the reaction fluid was examined on an HPLC system to examine the amount of acetyl phosphate and acetyl-CoA. The HPLC method was as follows: An Agilent 1200 HPLC was fitted with a HYPERSIL ODS COLUMN 150 mm×3 mm equipped with a BetaSil C18 20 mm×2.1 mm guard column. The column was heated to 25° C. with the sample block being maintained at 4° C. HPLC method comprised of 5 μl sample injection volume and an isocratic mobile phase comprised of 75 mM CH₃COONa (sodium acetate) and 100 mM NaH₂PO₄ (sodium dihydrogen phosphate) mixed with acetonitrile (ACN) in a ratio 94:6. The run time was a total of 12 minutes with acetyl-CoA eluting at 8.5 mins and coenzyme A (CoA) eluting at 3.9 minutes. A diode array detector (Agilent) was used for the detection of the molecule of interest at 259 nm.

2.5 Optimization of Acetyl-Coenzyme A C-acetyltransferase (PhaA): PhaA was mixed with epoxy methacrylate resin and the immobilized enzyme was used to convert acetyl-CoA into acetoacetyl-CoA. The reaction solution (50 mM Tris, 50 mM KH₂PO₄, 50 mM K₂HPO₄, PH 8.0, 5.0 mM MgCl₂, 100 mM NaCl, 2.5 mM acetyl COA) was mixed with immobilized enzyme at 32° C. for 8 hours. Immobilized PhaA (20 μM) was able to convert 44% of 2.5 mM acetyl-CoA for a yield of 1.1 mM (1.1 g/L) acetoacetyl-CoA. The retention time of AcCoA and acetoacetyl CoA coincide; therefore, PhaA activity was measured based on the amount of CoA produced in the reaction, as CoA and acetoacetyl COA are produced in equimolar amounts. For sampling, the reaction fluid was examined on an HPLC system to examine the amount of AcCoA and CoA. The HPLC method was as follows: An Agilent 1200 HPLC was fitted with a HYPERSIL ODS COLUMN 150 mm×3 mm equipped with a BetaSil C18 20 mm×2.1 mm guard column. The column was heated to 25° C. with the sample block being maintained at 4° C. HPLC method comprised of 5 μl sample injection volume and an isocratic mobile phase comprised of 75 mM CH₃COONa and 100 mM NaH₂PO₄ mixed with ACN in a ratio 94:6. The run time was a total of 12 minutes with acetyl-coA eluting at 8.5 mins and coenzyme A eluting at 3.9 minutes. A diode array detector (Agilent) was used for the detection of the molecule of interest at 259 nm.

2.6 Optimization of Hydroxymethylglutaryl-CoA synthase (HMGS): HMGS was mixed with epoxy methacrylate resin and the immobilized enzyme was used to convert acetoacetyl COA to HMG-CoA. The reaction solution (50 mM Tris 100 mM NaCl, 5 mM MgCl₂ pH 7.5, 5 mM acetoacetyl COA) was mixed with 0.5 μM immobilized enzyme at 32° C. for 2 hours. After 2 hours, the reaction solution was incubated with 20.7 μM HMGR and 5 mM NADPH. HMGR is used to convert NADPH into NADP+ and thus reaction performance can be monitored at 340 nm. The coupled reaction was able to convert 54% of the starting material to mevalonic acid (2.7 mM or 416 mg/L). The activity of HMGR was measured by monitoring the loss of NADPH at 340 nm using a spectrophotometer.

2.7 Optimization of Hydroxymethylglutaryl-CoA reductase (HMGR): HMGR was mixed with epoxy methacrylate resin and the immobilized enzyme was used to convert NADPH into NADP+. The reaction solution (50 mM Tris, 100 mM NaCl, 5 mM MgCl₂ PH 7.0, 5 mM NADPH) was mixed with 0.4 μM immobilized enzyme at 32° C. for 2 hours. Immobilized HMGR was able to convert 98% of 5 mM (nicotinamide adenine dinucleotide phosphate (NADPH) for a yield of 4.9 mM NADP+ which is equimolar to mevalonic acid produced in the reaction (4.9 mM or 755 mg/L). The activity of HMGR was measured by monitoring the loss of NADPH at 340 nm using a spectrophotometer.

2.8 Optimization of Mevalonate Kinase (MVK): MVK was mixed with macroporous polymethacrylate resin and the immobilized enzyme was used to convert mevalonic acid into mevalonic acid-5-phosphate. Reaction solution (50 mM Tris, 5 mM MgCl₂, PH 8, 4 mM ATP, 4 mM mevalonic acid) was mixed with 133 μM immobilized enzyme at 37° C. for 8 hours. Immobilized MVK was able to convert 79% of 4 mM ATP for a yield of 3.16 mM (1.68 g/L) ADP. For sampling, the reaction mixture was examined on an HPLC system to examine the amount of adenosine triphosphate (ATP) and adenosine diphosphate (ADP). The HPLC method was as follows: An Agilent 1200 HPLC was fitted with a HYPERSIL ODS COLUMN 150 mm×3 mm equipped with a BetaSil C18 20 mm×2.1 mm guard column. The column was heated to 25° C. with the sample block being maintained at 4° C. HPLC method comprised of 5 μl sample injection volume and an isocratic mobile phase comprised of 100 mM KH₂PO₄ (potassium dihydrogen phosphate), 8 mM TBAHS (tetrabutylammonium hydrogen sulfate), pH 6.0, 20% methanol (v/v). The run time was a total of 10 minutes with ATP eluting at 5.7 mins and ADP eluting at 4.6 minutes. A diode array detector (Agilent) was used for the detection of the molecule of interest at 254 nm.

2.9 Optimization of Phosphomevalonate Kinase (PMVK): PMVK was mixed with amino C₆ methacrylate resin and the immobilized enzyme was used to convert mevalonic acid-5-phosphate into mevalonic acid-5-pyrophosphate. The reaction solution (50 mM Tris, 5 mM MgCl₂, pH 8, 4 mM ATP, 4 mM mevalonic acid-5-phosphate) was mixed with 160 μM immobilized enzyme at 37° C. for 32 hours. Immobilized MVK was able to convert 96% of 4 mM ATP for a yield of 3.84 mM (1.79 g/L) ADP. For sampling, the reaction fluid was examined on an HPLC system to examine the amount of ATP and ADP. The HPLC method was as follows: An Agilent 1200 HPLC was fitted with a HYPERSIL ODS COLUMN 150 mm×3 mm equipped with a BetaSil C18 20 mm×2.1 mm guard column. The column was heated to 25° C. with the sample block being maintained at 4° C. HPLC method comprised of 5 μl sample injection volume and an isocratic mobile phase comprised of 100 mM KH₂PO₄, 8 mM TBAHS, pH 6.0, 20% methanol (v/v). The run time was a total of 10 minutes with ATP eluting at 5.7 mins and ADP eluting at 4.6 minutes. A diode array detector (Agilent) was used for the detection of the molecule of interest at 254 nm.

2.10 Optimization of Diphosphomevalonate Kinase (MDC): MDC was mixed with epoxy methacrylate resin and the immobilized enzyme was used to convert mevalonic acid-5-pyrophosphate into isopentenyl pyrophosphate. Reaction solution (50 mM Tris, 5 mM MgCl₂, pH 8, 4 mM ATP, 4 mM mevalonic acid-5-pyrophosphate) was mixed with 160 μM immobilized enzyme at 37° C. for 32 hours. Immobilized MVK was able to convert 94% of 2 mM ATP for a yield of 1.8 mM (839 mg/L) ADP. For sampling, the reaction fluid was examined on an HPLC system to examine the amount of ATP and ADP. The HPLC method was as follows: An Agilent 1200 HPLC was fitted with a HYPERSIL ODS COLUMN 150 mm×3 mm equipped with a BetaSil C18 20 mm×2.1 mm guard column. The column was heated to 25° C. with the sample block being maintained at 4° C. HPLC method comprised of 5 μl sample injection volume and an isocratic mobile phase comprised of 100 mM KH₂PO₄, 8 mM TBAHS, pH 6.0, 20% methanol (v/v). The run time was a total of 10 minutes with ATP eluting at 5.7 mins and ADP eluting at 4.6 minutes. A diode array detector (DAD) was used for the detection of the molecule of interest at 254 nm.

2.11 Optimization of Isopentenyl-diphosphate Delta-isomerase (IDI): IDI was mixed with macroporous polymethacrylate resin and the immobilized enzyme was used to convert isopentenyl pyrophosphate (IPP) into dimethylallyl pyrophosphate (DMAPP). The reaction solution (50 mM Tris pH 8, 5 mM MgCl₂, 10 mM NaCl, 0.24 mM IPP) was mixed with 86 μM immobilized enzyme at 25° C. for 2 hours. Then, the reaction mixture was incubated with 0.24 mM olivetolic acid, 85 μM NphB, and 29.7 μM FPPS for 2 hours. Completed reactions were extracted 3× with ethyl acetate, evaporated, and then resuspended in methanol for analysis on an HPLC system to examine the amount of CBGA present in the reaction mixture. The coupled reaction was able to convert 28% of the starting material to 21.3 mg/L of the product. The HPLC method was as follows: An Agilent 1200 HPLC was fitted with a 250 mm×4.6 mm, 5 μm liChrospher RP8 column equipped with a guard column. The column was heated to 30° C. with the sample block being maintained at 25° C. HPLC method comprised of 5 μl sample injection volume and an isocratic mobile phase comprised of 25% buffer A (water, 0.1% formic acid, 5 mM ammonium formate) and 75% buffer B (acetonitrile, 0.1% formic acid, 5 mM ammonium formate). CBGA produced in the reaction was measured using DAD at 228 nm. The run time was a total of 10 minutes with CBGA eluting at 3.68 mins.

2.12 Optimization of Polyprenyl synthetase family protein (FPPS): FPPS was mixed with macroporous polymethacrylate resin and the immobilized enzyme was used to convert isopentenyl pyrophosphate (IPP) and dimethylallyl pyrophosphate (DMAPP) into geranyl pyrophosphate (GPP). The reaction solution (50 mM Tris pH 8, 5 mM MgCl₂, 10 mM NaCl, 0.24 mM IPP, 0.24 mM DMAPP, 0.24 mM olivetolic acid (OA)) and 120 μM NphB protein was mixed with 86 μM immobilized enzyme at 25° C. for 4 hours. Immobilized FPPS was able to convert 81% of 240 μM IPP and 240 μM DMAPP for a yield of 195 μM GPP. Analysis of GPP production is coupled to the activity of the prenyltransferase (NphB) that combines GPP and olivetolic acid to produce CBGA. Completed reactions were extracted 3× with ethyl acetate, evaporated, and resuspended in methanol for analysis on an HPLC system to examine the amount of CBGA present in the reaction mixture. The HPLC method was as follows: An Agilent 1200 HPLC was fitted with a 250 mm×4.6 mm, 5 μm LiChrospher RP8 column equipped with a guard column. The column was heated to 30° C. with the sample block being maintained at 25° C. HPLC method comprised of 5 μl sample injection volume and an isocratic mobile phase comprised of 25% buffer A (water, 0.1% formic acid, 5 mM ammonium formate) and 75% buffer B (acetonitrile, 0.1% formic acid, 5 mM ammonium formate). The coupled reaction yielded cannabigerolic acid (CBGA) at 129.6 μM or 41 mg/L. CBGA produced in the reaction was measured using DAD at 228 nm. The run time was a total of 10 minutes with CBGA eluting at 3.68 mins.

2.13 Optimization of Polyphosphate kinase 2 (PPK2): PPK2 was mixed with epoxy methacrylate resin and the immobilized enzyme was used to convert ADP into ATP to recycle this cofactor. Reaction solution (10 mM Tris pH 9, 10 mM MgCl₂, 10 mM NaCl, 5.0 mM polyphosphate, 5.0 mM ADP) was mixed with 95 μM immobilized enzyme at 37° C. for 1 hour. Immobilized PPK2 was able to convert 5.0 mM ADP for a yield of 5.0 mM ATP (100%, 2.5 g/L). For sampling, the reaction fluid was examined on an HPLC system to examine the amount of ATP and ADP present in the reaction mixture. The HPLC method was as follows: An Agilent 1200 HPLC was fitted with a HYPERSIL ODS Column 150 mm×3 mm equipped with a BetaSil C18 20 mm×2.1 mm guard column. The column was heated to 25° C. with the sample block being maintained at 4° C. For each sample, 5 μL was injected and an isocratic mobile phase comprised of 100 mM KH₂PO₄, 8 mM TBAHS, pH 6.0, 20% methanol. The run time was a total of 10 minutes with ATP eluting at 5.7 mins and ADP eluting at 4.6 minutes. A diode array detector (DAD) was used for the detection of the molecule of interest at 254 nm.

2.14 Optimization of Glucose Dehydrogenase (GDH): GDH was mixed with epoxy methacrylate resin and the immobilized enzyme was used to convert NADP+ into NADPH to recycle this essential cofactor. The reaction solution (50 mM Tris pH 9, 20 mM glucose, 5.0 mM NADP+) was mixed with 200 μM immobilized enzyme at 22° C. for 15 minutes. Immobilized GDH was able to convert 5.0 mM NADP+ to yield 5.0 mM NADPH (100%, 3.7 g/L). The activity of GDH was detected by measuring the NADPH concentration of the reaction solution with a plate reader at 340 nm.

TABLE 2
Enzyme Sequences:

| | | SEQ |
|---|---|---|
| Enzyme: | Sequence: | ID NO: |
| Maltose Binding | KIEEGKLVIWINGDKGYNGLAEVGKKFEKDTGIKVTVEHPD | 1 |
| Protein Alditol | KLEEKFPQVAATGDGPDIIFWAHDRFGGYAQSGLLAEITPD | |
| oxidase (MPB-ALDO) | KAFQDKLYPFTWDAVRYNGKLIAYPIAVEALSLIYNKDLLP | |
| Streptomyces | NPPKTWEEIPALDKELKAKGKSALMFNLQEPYFTWPLIAAD | |
| coelicolor A3 | GGYAFKYENGKYDIKDVGVDNAGAKAGLTFLVDLIKNKHMN | |
| Accession: | ADTDYSIAEAAFNKGETAMTINGPWAWSNIDTSKVNYGVTV | |
| WP_011030685 | LPTFKGQPSKPFVGVLSAGINAASPNKELAKEFLENYLLTD | |
| | EGLEAVNKDKPLGAVALKSYEEELAKDPRIAATMENAQKGE | |
| | IMPNIPQMSAFWYAVRTAVINAASGRQTVDEALKDAQTRIT | |
| | KGENLYFQGGMASMTGGQQMGRGSGMSDITVTNWAGNITYT | |
| | AKELLRPHSLDALRALVADSARVRVLGSGHSFNEIAEPGDG | |
| | GVLLSLAGLPSVVDVDTAARTVRVGGGVRYAELARVVHARG | |
| | LALPNMASLPHISVAGSVATGTHGSGMGNGSLASMVREVEL | |
| | VTADGSTVVIARGDERFGGAVTSLGALGVVTSLTLDLEPAY | |
| | EMEQHVFTELPLAGLDPATFETVMAAAYSVSLFTDWRAPGF | |
| | RQVWLKRRTDRPLDGFPYAAPATEKMHPVPGMPAVNCTEQF | |
| | GVPGPWHERLPHFRAEFTPSSGAELQSEYLMPREHALAALH | |
| | AMDAIRETLAPVLQTCEIRTVAADAQWLSPAYGRDTVAAHF | |
| | TWVEDTAAVLPVVRRLEEALVPFAARPHWGKVFTVPAGELR | |
| | ALYPRLADFGALARALDPAGKFTNAFVRGVLAG | |
| Dihydroxy-acid | MAENWRSRIITEGVQRTPNRAMLRAVGFGDEDFNKPIVGVA | 2 |
| dehydratase (DHAD) | SAHSTITPCNMGIAALASRAEAGIRAAGGMPQLFGTITVSD | |
| Thermosynechococcus | GISMGTEGMKYSLVSRDVIADSIETVCNAQSMDGVLAIGGC | |
| vulcanus | DKNMPGAMIAMARMNIPAIFVYGGTIKPGHWQGQDLTVVSA | |
| Accession: | FEAVGQFSAGKMDEATLHAIEHHACPGAGSCGGMFTANTMS | |
| WP_126985616 | SAFEAMGMSLMYSSTMTAEDAEKADSTELAGKVLVEAIRKN | |
| | IRPRDIITRKSIENAISVIMAVGGSTNAVLHFLAIAHSAEV | |
| | PLTIDDFETIRQRVPVLCDLKPSGKYVTADLHRAGGIPQVM | |
| | KMLLNAGLLHGDCLTITGETIAERLRHVPDTPDPNQDVIRP | |
| | FDQPLYATGHLAILKGNLASEGAVAKISGVKNPQITGPARV | |
| | FDSEEACLDAILAGKINPGDVIVIRYEGPVGGPGMREMLAP | |
| | TSAIIGAGLGDSVGLITDGRFSGGTYGMVVGHVAPEAAVGG | |
| | TIALVQEGDSITIDAHRRLLQLNVSEEELAARRAKWQPPAP | |
| | RYTRGVLAKYAKLVSSSSLGAVTDRFV | |
| Phosphate | MTTDLFTALKAKVTGTARKIVFPEGTDDRILTAASRLATEQ | 3 |
| acetyltransferase | VLQPIVLGDEQAIRVKAAALGLPLEGVEIVNPRRYGGFDEL | |
| (PTA) | VSAFVERRKGKVTEETARELLFDENYFGTMLVYMGAADGLV | |
| Geobacillus | SGAAHSTADTVRPALQIIKTKPGVDKTSGVFIMVRGDEKYV | |
| stearothermophilus | FADCAINIAPNSHDLAEIAVESARTAKMFGLKPRVVLLSFS | |
| Accession: | TKGSASSPETEKVVEAVRLAKEMAPDLILDGEFQFDAAFVP | |
| WP_053532564 | EVAKKKAPDSVIQGDANVFIFPSLEAGNIGYKIAQRLGGFE | |
| | AVGPILQGLNKPVNDLSRGCSAEDAYKLALITAAQSLGE | |
| Acetyl-CoA C- | MTDVVIVSAARTAVGKFGGSLAKIPAPELGAVVIKAALERA | 4 |
| acetyltransferase | GVKPEQVSEVIMGQVLTAGSGQNPARQAAIKAGLPAMVPAM | |
| (PhaA) | TINKVCGSGLKAVMLAANAIMAGDAEIVVAGGQENMSAAPH | |
| Multispecies | VLPGSRDGFRMGDAKLVDTMIVDGLWDVYNQYHMGITAENV | |
| Cupriavidus | AKEYGITREAQDEFAVGSQNKAEAAQKAGKFDEEIVPVLIP | |
| Accession: | QRKGDPVAFKTDEFVRQGATLDSMSGLKPAFDKAGTVTAAN | |
| WP_010810132 | ASGLNDGAAAVVVMSAAKAKELGLTPLATIKSYANAGVDPK | |
| | VMGMGPVPASKRALSRAEWTPQDLDLMEINEAFAAQALAVH | |
| | QQMGWDTSKVNVNGGAIAIGHPIGASGCRILVTLLHEMKRR | |
| | DAKKGLASLCIGGGMGVALAVERK | |
| Hydroxymethyl- | MTIGIDKISFFVPPYYIDMTALAEARNVDPGKFHIGIGQDQ | 5 |
| glutaryl-CoA | MAVNPISQDIVTFAANAAEAILTKEDKEAIDMVIVGTESSI | |
| synthase | DESKAAAVVLHRLMGIQPFARSFEIKEGCYGATAGLQLAKN | |
| (HMGS_A110G) | HVALHPDKKVLVVAADIAKYGLNSGGEPTQGAGAVAMLVAS | |
| Enterococcus | EPRILALKEDNVMLTQDIYDFWRPTGHPYPMVDGPLSNETY | |
| faecalis | IQSFAQVWDEHKKRTGLDFADYDALAFHIPYTKMGKKALLA | |
| Accession: | KISDQTEAEQERILARYEESIIYSRRVGNLYTSSLYLGLIS | |
| WP_010785222 | LLENATTLTAGNQIGLFSYGSGAVAEFFTGELVAGYQNHLQ | |
| | KETHLALLDNRTELSIAEYEAMFAETLDTDIDQTLEDELKY | |
| | SISAINNTVRSYRN | |
| Hydroxymethyl- | MKTVVIIDALRTPIGKYKGSLSQVSAVDLGTHVTTQLLKRH | 6 |
| glutaryl-CoA | STISEEIDQVIFGNVLQAGNGQNPARQIAINSGLSHEIPAM | |
| reductase | TVNEVCGSGMKAVILAKQLIQLGEAEVLIAGGIENMSQAPK | |
| (HMGR) | LQRFNYETESYDAPFSSMMYDGLTDAFSGQAMGLTAENVAE | |
| Enterococcus | KYHVTREEQDQFSVHSQLKAAQAQAEGIFADEIAPLEVSGT | |
| faecalis | LVEKDEGIRPNSSVEKLGTLKTVFKEDGTVTAGNASTINDG | |
| Accession: | ASALIIASQEYAEAHGLPYLAIIRDSVEVGIDPAYMGISPI | |
| WP_002361742 | KAIQKLLARNQLTTEEIDLYEINEAFAATSIVVQRELALPE | |
| | EKVNIYGGGISLGHAIGATGARLLTSLSYQLNQKEKKYGVA | |
| | SLCIGGGLGLAMLLERPQQKKNSRFYQMSPEERLASLLNEG | |
| | QISADTKKEFENTALSSQIANHMIENQISETEVPMGVGLHL | |
| | TVDETDYLVPMATEEPSVIAALSNGAKIAQGFKTVNQQRLM | |
| | RGQIVFYDVADAESLIDELQVRETEIFQQAELSYPSIVKRG | |
| | GGLRDLQYRAFDESFISVDFLVDVKDAMGANIVNAMLEGVA | |
| | ELFREWFAEQKILFSILSNYATESVVTMKTAIPVSRLSKGS | |
| | NGREIAEKIVLASRYASLDPYRAVTHNKGIMNGIEAVVLAT | |
| | GNDTRAVSASCHAFAVKEGRYQGLTSWTLDGEQLIGEISVP | |
| | LALATVGGATKVLPKSQAAADLLAVTDAKELSRVVAAVGLA | |
| | QNLAALRALVSEGIQKGHMALQARSLAMTVGATGKEVEAVA | |
| | QQLKRQKTMNQDRALAILNDLRKQ | |
| Mevalonate Kinase | MLKFSKIEKLLRNNMVSCSAPGKIYLFGEHAVVYGETAIAC | 7 |
| (MVK) | AVELRTRVRAELNDSITIQSQIGRTGLDFEKHPYVSAVIEK | |
| Methanosarcina | MRKSIPINGVFLTVDSDIPVGSGLGSSAAVTIASIGALNEL | |
| mazei Tuc01 | FGFGLSLQEIAKLGHEIElKVQGAASPTDTYVSTFGGVVTI | |
| Accession: | PERRKLKTPDCGIVIGDTGVFSSTKELVANVRQLRESYPDL | |
| AGF97182 | IEPLMTSIGKISRIGEQLVLSGDYASIGRLMNVNQGLLDAL | |
| | GVNILELSQLIYSARAAGAFGAKITGAGGGGCMVALTAPEK | |
| | CNQVAEAIAGAGGKVTITKPTEQGLKVD | |
| Phosphomevalonate | MIAVKTCGKLYWAGEYAILEPGQLALIKDIPIYMRAEIAFS | 8 |
| Kinase (PMVK) | DSYRIYSDMFDFAVDLRPNPDYSLIQETIALMGDFLAVRGQ | |
| Streptococcus | NLRPFSLAIYGKMEREGKKFGLGSSGSVVVLVVKALLALYN | |
| pneumoniae | LSVDQNLLFKLTSAVLLKRGDNGSMGDLACIAAEDLVLYQS | |
| Accession: | FDRQKVAAWLEEENLATVLERDWGFSISQVKPTLECDFLVG | |
| WP_000562411 | WTKEVAVSSHMVQQIKQNINQNFLTSSKETVVSLVEALEQG | |
| | KSEKIIEQVEVASKLLEGLSTDIYTPLLRQLKEASQDLQAV | |
| | AKSSGAGGGDCGIALSFDAQSTKTLKNRWADLGIELLYQER | |
| | IGHDDKS | |
| Diphosphomevalonate | MYHSLGNQFDTRTRTSRKIRRERSCSDMDREPVTVRSYANI | 9 |
| Kinase (MDC) | AIIKYWGKKKEKEMVPATSSISLTLENMYTETTLSPLPANV | |
| Streptococcus | TADEFYINGQLQNEVEHAKMSKIIDRYRPAGEGFVRIDTQN | |
| pneumoniae R6 | NMPTAAGLSSSSSGLSALVKACNAYFKLGLDRSQLAQEAKF | |
| Accession: | ASGSSSRSFYGPLGAWDKDSGEIYPVETDLKLAMIMLVLED | |
| AAK99143 | KKKPISSRDGMKLCVETSTTFDDVVVRQSEKDYQDMLIYLK | |
| | ENDFAKIGELTEKNALAMHATTKTASPAFSYLTDASYEAMD | |
| | FVRQLREKGEACYFTMDAGPNVKVFCQEKDLEHLSEIFGQR | |
| | YRLIVSKTKDLSQDDCC | |
| Isopentenyl- | MQTEHVILLNAQGVPTGTLEKYAAHTADTRLHLAFSSWLFN | 10 |
| diphosphate Delta- | AKGQLLVTRRALSKKAWPGVWTNSVCGHPQLGESNEDAVIR | |
| isomerase (IDI) | RCRYELGVEITPPESIYPDFRYRATDPSGIVENEVCPVFAA | |
| Multispecies | RTTSALQINDDEVMDYQWCDLADVLHGIDATPWAFSPWMVM | |
| Bacteria | QATNREARKRLSAFTQLK | |
| Accession: | | |
| WP_001192820 | | |
| Polyprenyl | MAQLSVEQFLNEQKQAVETALSRYIERLEGPAKLKKAMAYS | 11 |
| synthetase family | LEAGGKRIRPLLLLSTVRALGKDPAVGLPVACAIEMIHTYF | |
| protein (FPPS S82F) | LIHDDLPSMDNDDLRRGKPTNHKVFGEAMAILAGDGLLTYA | |
| Geobacillus | FQLITEIDDERIPPSVRLRLIERLAKAAGPEGMVAGQAADM | |
| stearothermophilus | EGEGKTLTLSELEYIHRHKTGKMLQYSVHAGALIGGADARQ | |
| Accession: | TRELDEFAAHLGLAFQIRDDILDIEGAEEKIGKPVGSDQSN | |
| WP_033016440 | NKATYPALLSLAGAKEKLAFHIEAAQRHLRNADVDGAALAY | |
| | ICELVAARDH | |
| Polyphosphate | MALDEAPAEARPGSRAVELEIDGRSRIFDIDDPDLPKWIDE | 12 |
| kinse 2 (PPK2) | EAFRSDDYPYKKKLDREEYEETLTKLQIELVKVQFWMQATG | |
| Rhizobacteria | KRVMAVFEGRDAAGKGGAIHATTANMNPRSARVVALTKPTE | |
| Accession: | TERGQWYFQRYVATFPTAGEFVLFDRSWYNRAGVEPVMGFC | |
| WP_010968631 | TPDQYEQFLKEAPRFEEMIANEGIHLFKFWINIGREMQLKR | |
| | FHDRRHDPLKIWKLSPMDIAALSKWDDYTGKRDRMLKETHT | |
| | EHGPWAVIRGNDKRRSRINVIRHMLTKLDYDGKDEAAIGEV | |
| | DEKILGSGPGFLR | |
| Glucose | MYSDLEGKWVITGSASGLGRAMGVRFAREKAKWINYRSRES | 13 |
| Dehydrogenase | EANDVLEEIKKVGGEAIAVKGDVTVESDVVNLIQSAVKEFG | |
| (GDH) | TLDVMINNAGIENAVPSHEMPLEDWNRVINTNLTGAFLGSR | |
| Bacillus sp. G3 | EAIKYFVEHDIKGSVINMSSVHEKIPWPLFVHYAASKGGMK | |
| Accession: | LMTETLAMEYAPKGIRVNNIGPGAINTPINAEKFADPKKRA | |
| GQ402830.1 | DVESMIPMGYIGKPEEIAAVATWLASSEASYVTGITLFADG | |
| | GMTLYPSFQAGRG | |

*Note:
Pyruvate Oxidase (PyOx, Aerococcus viridans) was purchased from AG Scientific, product P-1600.

3.0 Use of all immobilized Enzymes to create GPP from glycerol: After demonstrating generation of GPP from glycerol with free enzymes, and also demonstrating that all of the required individual enzymes are active when immobilized, the next aim was to generate GPP from glycerol using immobilized enzymes. Each enzyme was immobilized onto 10 mg of resin as listed in Table 3. Purified enzymes were mixed with resin for 18 hours at 21° C. and immobilized enzymes were pooled into a single tube.

TABLE 3
Specifics for immobilized enzyme batch reactions.

| | Amount | |
|---|---|---|
| Enzyme Name | (mg) | Resin |
| MBP-Aldo (Aldo) | 0.5 | Amino C₆ methacrylate |
| Dihydroxy Acid Dehydratase (DHAD) | 0.5 | Amino C₆ methacrylate |
| Pyruvate Oxidase (PyOx) | 0.25 | Amino C₆ methacrylate |
| Acetyl-phosphate transferase (PTA) | 0.15 | Epoxy methacrylate |
| Acetyl-CoA acetyltransferase (PhaA) | 0.1 | Epoxy methacrylate |
| HMG-CoA Synthase A110G (HMGS) | 0.15 | Epoxy methacrylate |
| HMG-CoA Reductase (HMGR) | 0.4 | Epoxy methacrylate |
| Mevalonate Kinase (MVK) | 0.2 | Macroporous |
| | | polymethacrylate |
| Phosphomevalonate Kinase (PMVK) | 0.2 | Amino C₆ methacrylate |
| Diphosphomevalonate Kinase (MDC) | 0.4 | Epoxy methacrylate |
| Isopentyl-PP Isomerase (IDI) | 0.35 | Macroporous |
| | | polymethacrylate |
| Farnesyl-PP synthase S82F (FPPS) | 0.2 | Macroporous |
| | | polymethacrylate |
| Prenyl Transferase (NphB) | 0.2 | Macroporous |
| | | polymethacrylate |

For this multi-step reaction, the first three enzymes (Aldo, DHAD, and PyOX) were first added sequentially into the batch reactor. First, immobilized MBP-Aldo was added to reaction solution (50 mM Tris pH 9, 2.5 mM MgCl₂, 10 mM glycerol) for 18 h at 37° C. Next, immobilized DHAD was added to the reaction solution and incubated for 18 h at 45° C. The supernatant was removed from the immobilized enzymes, and the reaction solution was adjusted to contain 50 mM NaCl, 20 mM potassium phosphate (pH 6.5), 10 mM thiamine pyrophosphate, and finally, the pH of the reaction solution was adjusted to pH 6.0. Immobilized PyOx was then added to the reaction solution for 16 h at 37° C. The supernatant was then removed from immobilized enzyme and the reaction solution was adjusted to contain 50 mM Tris, 20 mM potassium phosphate, 2.5 mM MgCl₂, 50 mM NaCl, 10 mM NADPH, 10 mM ATP, 10 mM CoA, 4 mM olivetolic acid, and pH 8.0 for a final volume of 1.0 mL. The remaining ten immobilized enzymes in this pathway were added to the reaction mixture. The reactions were carried out for five days at 37° C. and were then extracted ethyl acetate (2×200 μL), evaporated under reduced pressure, and resuspended in methanol (1 mL) for analysis on a HPLC system to examine the amount of CBGA present in the reaction mixture. The HPLC method was as follows: An Agilent 1200 HPLC was fitted with a 250 mm×4.6 mm, 5 μm liChrospher RP8 column equipped with a guard column. The column was heated to 30° C. with the sample block being maintained at 25° C. HPLC method comprised of 5 μl sample injection volume and an isocratic gradient comprised of 25% buffer A (water, 0.1% formic acid, 5 mM ammonium formate) and 75% buffer B (acetonitrile, 0.1% formic acid, 5 mM ammonium formate) was used as the mobile phase. The reaction yielded cannabigerolic acid (CBGA) at 155 μM or 49 mg/L (FIGS. 2A and 2B). CBGA produced in the reaction was measured using DAD at 228 nm. The run time was a total of 10 minutes with CBGA eluting at 3.68 minutes.

4.0 Use of Immobilized Enzymes to create GPP from Glycerol with Cofactors (ATP and NADPH recycling): In addition to demonstrating the immobilized glycerol to GPP pathway success, the recycling of two common cofactors (ATP and NADPH) in the reaction was achieved. The immobilized enzyme composition shown in Table 4 was used in the reaction.

TABLE 4
Specifics for immobilized
cofactor recycling enzymes for batch reactions.

| | Amount | |
|---|---|---|
| Enzyme Name | (mg) | Resin |
| Polyphosphate kinase 2 (PPK2) | 0.1 | Epoxy methacrylate |
| Glucose dehydrogenase (GDH) | 0.1 | Epoxy methacrylate |
| MBP-Aldo (Aldo) | 0.5 | Amino C₆ methacrylate |
| Dihydroxy Acid Dehydratase (DHAD) | 0.5 | Amino C₆ methacrylate |
| Pyruvate Oxidase (PyOx) | 0.25 | Amino C₆ methacrylate |
| Acetyl-phosphate transferase (PTA) | 0.15 | Epoxy methacrylate |
| Acetyl-CoA acetyltransferase (PhaA) | 0.1 | Epoxy methacrylate |
| HMG-CoA Synthase A110G (HMGS) | 0.15 | Epoxy methacrylate |
| HMG-CoA Reductase (HMGR) | 0.4 | Epoxy methacrylate |
| Mevalonate Kinase (MVK) | 0.2 | Macroporous |
| | | polymethacrylate |
| Phosphomevalonate Kinase (PMVK) | 0.2 | Amino C₆ methacrylate |
| Diphosphomevalonate Kinase (MDC) | 0.4 | Epoxy methacrylate |
| lsopentyl-PP Isomerase (IDI) | 0.35 | Macroporous |
| | | polymethacrylate |
| Farnesyl-PP synthase S82F (FPPS) | 0.2 | Macroporous |
| | | polymethacrylate |
| Prenyl Transferase (NphB) | 0.2 | Macroporous |
| | | polymethacrylate |

For this multi-step reaction, the first three enzymes (Aldo, DHAD, and PyOX) were first added sequentially into the batch reactor. First, immobilized MBP-Aldo was added to reaction solution (50 mM Tris pH 9, 2.5 mM MgCl₂, 10 mM glycerol) for 18 h at 37° C. Next, immobilized DHAD was added to the reaction solution and incubated for 18 h at 45° C. The supernatant was removed from the immobilized enzymes, and the reaction solution was adjusted to contain 50 mM NaCl, 20 mM potassium phosphate (pH 6.5), 10 mM thiamine pyrophosphate, and finally, the pH of the reaction solution was adjusted to pH 6.0. Immobilized PyOx was then added to the reaction solution for 16 h at 37° C. The supernatant was then removed from immobilized enzyme and the reaction solution was adjusted to contain 50 mM Tris, 20 mM potassium phosphate, 2.5 mM MgCl₂, 50 mM NaCl, 10 mM NADPH, 10 mM ATP, 10 mM CoA, 4 mM olivetolic acid, and pH 8.0 for a final volume of 1.0 mL. The remaining 12 enzymes (as shown in Table 4) were added to a reaction solution of 50 mM Tris, 20 mM potassium phosphate, 2.5 mM MgCl₂, 50 mM NaCl, 3.3 mM NADPH, 3.3 mM ATP, 5.0 mM poly-phosphate, 5.0 mM glucose 10 mM CoA, 4 mM olivetolic acid and pH 8.0 in a final volume of 1.0 mL. Batch reactions continued for 5 days at 37° C. and were then extracted with ethyl acetate (3×200 μL), evaporated under reduced pressure, and resuspended in methanol (1 mL) for analysis on a HPLC system to examine the amount of CBGA present in the reaction mixture. The HPLC method was as follows: An Agilent 1200 HPLC was fitted with a 250 mm×4.6 mm, 5 μm liChrospher RP8 column equipped with a guard column. The column was heated to 30° C. with the sample block being maintained at 25° C. The HPLC method comprised of a 5 μl sample injection volume and a mobile phase comprised of 25% buffer A (water, 0.1% formic acid, 5 mM ammonium formate) and 75% buffer B (acetonitrile, 0.1% formic acid, 5 mM ammonium formate). The reaction yielded cannabigerolic acid (CBGA) at 114 μM or 36 mg/L. CBGA produced in the reaction was measured using DAD at 228 nm. The run time was a total of 10 minutes with CBGA eluting at 3.68 mins.

### Embodiments

The following embodiments are intended to be illustrative only and not to be limiting in any way.

Embodiment 1: A method of converting glycerol to geranyl pyrophosphate (GPP) and additional secondary metabolites, the method comprising (a) adding glycerol to a reaction mixture; (b) adding a plurality of enzymes to the reaction mixture from (a), wherein the enzymes are selected from a group consisting of alditol oxidase (Aldo), dihydroxy-acid dehydratase (DHAD), pyruvate oxidase (PyOx), acetyl-phosphate transferase (PTA), acetyl-CoA acetyltransferase (PhaA), HMG-COA Synthase A110G (HMGS), HMG-CoA Reductase (HMGR), mevalonate kinase (MVK), phosphomevalonate kinase (PMVK), diphosphomevalonate kinase (MDC), isopentyl-PP Isomerase (IDI), and farnesyl-PP synthase S82F (FPPS); (c) removing a supernatant from the reaction mixture from (b); and isolating or producing GPP.

Embodiment 2: The method of embodiment 1, wherein at least two enzymes are added to the reaction mixture.

Embodiment 3: The method of embodiment 1 or embodiment 2, wherein at least five enzymes are added to the reaction mixture.

Embodiment 4: The method of any one of embodiments 1-3, wherein at least ten enzymes are added to the reaction mixture.

Embodiment 5: The method of any one of embodiments 1-4, further comprising adding a NphB enzyme to step (b) to convert GPP to (cannabigerolic acid) CBGA

Embodiment 6: The method of embodiment 5, wherein the conversion of GPP to CBGA is used to determine the amount of GPP produced from the method.

Embodiment 7: The method of any one of embodiments 1-6, wherein the reaction mixture comprises cofactors.

Embodiment 8: The method of embodiment 7, wherein the cofactors are adenosine triphosphate (ATP), nicotinamide adenine dinucleotide (NAD⁺), nicotinamide adenine dinucleotide phosphate (NADP⁺), or a combination thereof.

Embodiment 9: The method of embodiment 7 or embodiment 8, wherein the cofactor are recycled.

Embodiment 10: The method of embodiment 9, wherein glucose dehydrogenase (GDH), and polyphosphate kinase 2 (PPK2) are added to the reaction mixture to recycle the cofactors.

Embodiment 11: The method of claim any one of embodiments 1-10, wherein one or more of the enzymes are immobilized.

Embodiment 12: The method of claim any one of embodiments 1-10, wherein the plurality of enzymes are immobilized

Embodiment 13: The method of claim any one of embodiments 1-10, wherein one or more of the enzymes are non-immobilized.

Embodiment 14: The method of claim any one of embodiments 1-10, wherein the plurality of enzymes are non-immobilized.

Embodiment 15: A method of converting glycerol to geranyl pyrophosphate (GPP) and additional secondary metabolites, the method comprising (a) adding glycerol and alditol oxidase (Aldo) to a reaction mixture; (b) adding dihydroxy-acid dehydratase (DHAD) to the reaction mixture from (a); (c) removing a supernatant of the reaction mixture from (b); (d) adding pyruvate oxidase (PyOx) to the supernatant of the reaction mixture from (c); (e) removing a supernatant of the reaction mixture from (d); (f) adding at least two enzymes selected from a group consisting of acetyl-phosphate transferase (PTA), acetyl-CoA acetyltransferase (PhaA), HMG-COA Synthase A110G (HMGS), HMG-CoA Reductase (HMGR), mevalonate kinase (MVK), phosphomevalonate kinase (PMVK), diphosphomevalonate kinase (MDC), isopentyl-PP Isomerase (IDI), farnesyl-PP synthase S82F (FPPS), and prenyl transferase (NphB) to the supernatant of the reaction mixture from (e); (g) removing a supernatant from the reaction mixture from (f); and (h) isolating GPP.

Embodiment 16: The method of embodiment 15 further comprising adding a NphB enzyme to step (f) to convert GPP to (cannabigerolic acid) CBGA.

Embodiment 17: The method of embodiment 16, wherein the conversion of GPP to CBGA is used to determine the amount of GPP produced from the method.

Embodiment 18: The method of any one of embodiments 15-17, wherein the reaction mixture comprises cofactors.

Embodiment 19: The method of embodiment 18, wherein the cofactors are adenosine triphosphate (ATP), nicotinamide adenine dinucleotide (NAD+), nicotinamide adenine dinucleotide phosphate (NADP+), or a combination thereof.

Embodiment 20: The method of embodiment 17 or embodiment 18, wherein the cofactor are recycled.

Embodiment 21: The method of embodiment 20, wherein glucose dehydrogenase (GDH), and polyphosphate kinase 2 (PPK2) are added to the reaction mixture to recycle the cofactors.

Embodiment 22: The method of claim any one of embodiments 15-21, wherein one or more of the enzymes are immobilized.

Embodiment 23: The method of claim any one of embodiments 15-21, wherein the plurality of enzymes are immobilized

Embodiment 24: The method of claim any one of embodiments 15-21, wherein one or more of the enzymes are non-immobilized.

Embodiment 25: The method of claim any one of embodiments 15-21, wherein the plurality of enzymes are non-immobilized.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-11. (canceled)

12. A method of converting glycerol to geranyl pyrophosphate (GPP) and additional secondary metabolites, the method comprising:
a) adding glycerol and alditol oxidase (Aldo) to a reaction mixture;
b) adding dihydroxy-acid dehydratase (DHAD) to the reaction mixture from (a);
c) removing a supernatant of the reaction mixture from (b);
d) adding pyruvate oxidase (PyOx) to the supernatant of the reaction mixture from (c);
e) removing a supernatant of the reaction mixture from (d);
f) adding at least two enzymes selected from a group consisting of acetyl-phosphate transferase (PTA), acetyl-CoA acetyltransferase (PhaA), HMG-COA Synthase A110G (HMGS), HMG-COA Reductase (HMGR), mevalonate kinase (MVK), phosphomevalonate kinase (PMVK), diphosphomevalonate kinase (MDC), isopentyl-PP Isomerase (IDI), and farnesyl-PP synthase S82F (FPPS) to the supernatant of the reaction mixture from (e);
g) removing a supernatant from the reaction mixture from (f); and
h) isolating GPP.

13. The method of claim 12 further comprising adding a NphB enzyme to step (f) to convert GPP to (cannabigerolic acid) CBGA.

14. The method of claim 13, wherein the conversion of GPP to CBGA is used to determine the amount of GPP produced from the method.

15. The method of claim 12, wherein the reaction mixture comprises cofactors.

16. The method of claim 15, wherein the cofactors are adenosine triphosphate (ATP), nicotinamide adenine dinucleotide (NAD⁺), nicotinamide adenine dinucleotide phosphate (NADP⁺), or a combination thereof.

17. The method of claim 15, wherein the cofactor are recycled.

18. The method of claim 17, wherein glucose dehydrogenase (GDH), and polyphosphate kinase 2 (PPK2) are added to the reaction mixture to recycle the cofactors.

19. The method of claim 15, wherein one or more of the enzymes are immobilized.

20. The method of claim 15, wherein one or more of the enzymes are non-immobilized.
